(19) United States
(12) Patent Application Publication
(10) Pub. No.: US 2017/0064249 A1
(43) Pub. Date: Mar. 2, 2017

(54) ENDOSCOPE IMAGING APPARATUS AND ENDOSCOPE

(71) Applicant: FUJIFILM Corporation, Tokyo (JP)

(72) Inventor: Ryou KITANO, Kanagawa (JP)

(73) Assignee: FUJIFILM Corporation, Tokyo (JP)

(21) Appl. No.: 15/249,458

(22) Filed: Aug. 28, 2016

(30) Foreign Application Priority Data
Aug. 31, 2015 (JP) 2015-171434

(51) Int. Cl. H04N 7/10; A61B 1/05; A61B 1/00; H04N 5/225

(52) U.S. Cl. CPC H04N 7/10; H04N 5/2253; H04N 5/2252; A61B 1/051; A61B 1/00124; H04N 2005/2255; H04N 5/23293

(57) ABSTRACT

An imaging apparatus includes: an image sensor having a photodetecting surface which crosses a longitudinal axis of an insertion unit of an endoscope; and a circuit board which is mounted with the image sensor and includes: a sensor mounting portion which has plural sensor connection lands to which respective terminals of the image sensor are connected and faces a back surface, opposite to the photodetecting surface, of the image sensor; and a polygonal-prism-shaped cable connection portion which has plural cable connection lands being electrically continuous with the respective sensor connection lands and extends parallel with the longitudinal axis, wherein an outline of the cable connection portion is smaller than an outline of the sensor mounting portion when viewed from a distant point on an axis of the cable connection portion, and the cable connection lands are formed on plural side surfaces of the cable connection portion in a distributed manner.

### CROSS-REFERENCE TO RELATED APPLICATIONS

This application claims the benefit of Japanese Patent Application JP 2015-171434, filed Aug. 31, 2015, the entire content of which is hereby incorporated by reference, the same as if set forth at length.

### FIELD OF THE INVENTION

The present invention relates to an endoscope and an endoscope imaging apparatus which is installed in a tip portion of an insertion unit of the endoscope.

### BACKGROUND OF THE INVENTION

JP-A-7-318815 discloses an endoscope imaging apparatus which is equipped with an image sensor and a circuit board which is mounted with the image sensor and to which plural cables are connected. The photodetecting surface of the image sensor is oriented perpendicularly to the longitudinal axis of the insertion unit. The circuit board is disposed on the back side, opposite to the photodetecting surface, of the image sensor and shaped like a polygonal prism that extends in the longitudinal axis of the insertion unit.

The circuit board is formed with plural lands that are connected to plural respective terminals projecting from the back surface of the image sensor and plural lands that are connected to the plural respective cables. These lands are arranged on (distributed to) the plural side surfaces of the polygonal-prism-shaped circuit board. In this manner, the circuit board is miniaturized and the outline of the circuit board is smaller than that of the image sensor when viewed from a distant point on the longitudinal axis of the insertion unit.

### SUMMARY OF THE INVENTION

The insertion units of endoscopes are required to be reduced in diameter and imaging apparatus installed in tip portions of the insertion units are also required to be reduced in diameter accordingly. In imaging apparatus in which the photodetecting surface of the image sensor is oriented perpendicularly to the longitudinal axis of the insertion unit as in the endoscope imaging apparatus disclosed in JP-A-7-318815, it is important that the image sensor be miniaturized; for example, there is demand for very small image sensors whose outlines are contained in a 1-mm square, for example.

To miniaturize imaging apparatus, it is essential to miniaturize the circuit board which is mounted with the image sensor. However, as the circuit board is miniaturized, the cables that are connected to the circuit board are made thinner and the lands, connected to the cables, of the circuit board are made smaller, which may lower the durability of the connections between the circuit board and the cables.

As for the connections between the circuit board and the cables, in the endoscope imaging apparatus disclosed in JP-A-7-318815, since the lands to which the cables are connected are arranged on (distributed to) the plural side surfaces of the polygonal-prism-shaped circuit board, it is possible to avoid size reduction of the lands and thereby prevent reduction in the durability of the connections between the circuit board and the cables.

However, usually, the image sensor is mounted on one surface of the circuit board. Also in the endoscope imaging apparatus disclosed in JP-A-7-318815, the lands to which the terminals of the image sensor are connected are formed in a concentrated manner on an end portion of one side surface of the polygonal-prism-shaped circuit board. Therefore, in the circuit board of the endoscope imaging apparatus disclosed in JP-A-7-318815, it is unavoidable to reduce the sizes of the lands to which the terminals of the image sensor are connected as the circuit board is miniaturized. This may lower the durability of the connections between the circuit board and the cables.

The present invention has been made in view of the above circumstances, and an object of the invention is therefore to increase the durability of both of the connections between an image sensor and a circuit board of an endoscope imaging apparatus that is installed in a tip portion of an insertion unit of an endoscope and the connections between the circuit board and cables, and to enable miniaturization of the endoscope imaging apparatus.

According to an aspect of the invention, there is provided an endoscope imaging apparatus which is installed in a tip portion of an insertion unit of an endoscope, comprising: an image sensor having a photodetecting surface which crosses the longitudinal axis of the insertion unit; and a circuit board which is mounted with the image sensor and comprises: a sensor mounting portion which has plural sensor connection lands to which respective terminals of the image sensor are connected and faces a back surface, opposite to the photodetecting surface, of the image sensor; and a polygonal-prism-shaped cable connection portion which has plural cable connection lands being electrically continuous with the respective sensor connection lands and extends parallel with the longitudinal axis, wherein an outline of the cable connection portion is smaller than an outline of the sensor mounting portion when viewed from a distant point on the axis of the cable connection portion, and the cable connection lands are formed on plural side surfaces of the cable connection portion in a distributed manner.

An endoscope according to another aspect of the invention comprises any of the above endoscope imaging apparatus.

The invention makes it possible to increase the durability of both of the connections between an image sensor and a circuit board of an endoscope imaging apparatus that is installed in a tip portion of an insertion unit of an endoscope and the connections between the circuit board and cables, and to enable miniaturization of the endoscope imaging apparatus.

### BRIEF DESCRIPTION OF THE DRAWINGS

FIG. 1 shows the configuration of an example endoscope system for description of an embodiment of the present invention.

FIG. 2 is a sectional view of a tip portion of an insertion unit of an endoscope shown in FIG. 1.

FIG. 3 is a perspective view of an imaging apparatus which is installed in the tip portion shown in FIG. 2.

FIG. 4 is a perspective view of an image sensor, a lens barrel, and a holder of the imaging apparatus of FIG. 3.

FIG. 5 is a perspective view of a circuit board of the imaging apparatus of FIG. 3.

FIG. 6 is another perspective view of the circuit board of the imaging apparatus of FIG. 3.

FIG. 7 is a rear view of the imaging apparatus of FIG. 3.

### DESCRIPTION OF SYMBOLS

1: Endoscope system
2: Endoscope
3: Light source unit
4: Processor unit
5: Monitor
6: Insertion unit
7: Manipulation unit
8: Universal cord
9: Connectors
10: Tip portion
11: Bendable portion
12: Soft portion
14: Treatment tool channel
20: Imaging apparatus
21: Image sensor
21a: Photodetecting surface
22: Lens barrel
23: Holder
24: Circuit board
25: Tip hard portion
26: Terminals
27: Cables
30: Sensor mounting portion
31: Cable connection portion
32: Sensor connection lands
33: Cable connection lands

### DETAILED DESCRIPTION OF THE INVENTION

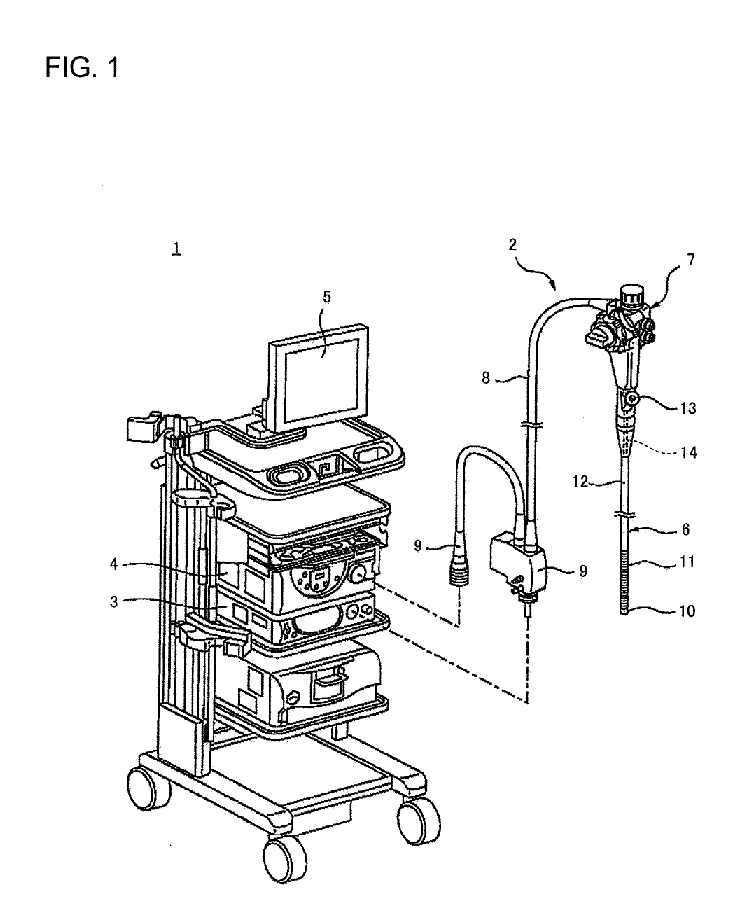

FIG. 1 shows an example endoscope system 1 for description of an embodiment of the present invention. The endoscope system 1 is composed of an endoscope 2, a light source unit 3, and a processor unit 4. The endoscope 2 is equipped with an insertion unit 6 to be inserted into a subject body, a manipulation unit 7 which is continuous with the insertion unit 6, and a universal cord 8 which extends from the manipulation unit 7. The insertion unit 6 is composed of a tip portion 10, a bendable portion 11 which is continuous with the tip portion 10, and a soft portion 12 which links the bendable portion 11 to the manipulation unit 7.

The tip portion 10 is equipped with an illumination optical system for emitting illumination light for illumination of an observation part, an imaging apparatus and an imaging optical system for shooting the observation part, and other components. The bendable portion 11 is configured so as to be bendable perpendicularly to the longitudinal axis of the insertion unit 6, and is bent by manipulating the manipulation unit 7. The soft portion 12 is configured so as to be relatively flexible, that is, flexible enough to deform so as to conform to the shape of an insertion route of the insertion unit 6.

The manipulation unit 7 is equipped with buttons for manipulating an imaging operation of the imaging apparatus installed in the tip portion 10 and a rotary knob for making a manipulation for bending the bendable portion 11. The manipulation unit 7 is formed with an insertion inlet 13 through which a treatment tool such as an electric scalpel is to be inserted, and a treatment tool channel 14 through which a treatment tool is to be inserted is formed in the insertion unit 6 so as to extend from the insertion inlet 13 to the tip portion 10.

Connectors 9 are provided at an intermediate position and one end of the universal cord 8. The endoscope 2 is connected, via the connector(s) 9, to the light source unit 3 for generating illumination light to be emitted from the illumination optical system provided in the tip portion 10 and the processor unit 4 for processing a video signal acquired by the imaging apparatus provided in the tip portion 10. The processor unit 4 generates video data of an observation part by processing a received video signal and displays the generated video data on a monitor 5. Furthermore, the processor unit 4 has the generated video data recorded.

A light guide and cables are disposed inside the insertion unit 6, the manipulation unit 7, and the universal cord 8. Illumination light generated by the light source unit 3 is guided by the light guide to the illumination optical system provided in the tip portion 10, and signals and power are transmitted between the imaging apparatus provided in the tip portion 10 and the processor unit 4 by the cables.

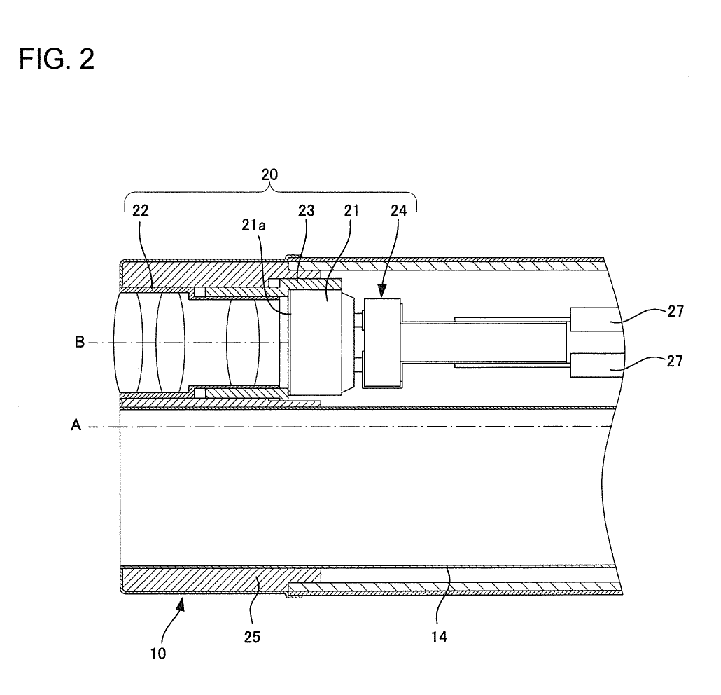

FIG. 2 shows the internal configuration of the tip portion 10 of the insertion unit 6. The tip portion 10 is equipped with an imaging apparatus 20 and a tip portion of the treatment tool channel 14. Although not shown in FIG. 2, the illumination optical system for emitting illumination light that is guided from the light source unit 3 by the light guide and other components are disposed in the tip portion 10.

The imaging apparatus 20 is equipped with an image sensor 21 such as a CCD (charge-coupled device) image sensor or a CMOS (complementary metal-oxide-semiconductor) image sensor, a lens barrel 22 which houses an imaging optical system for forming a subject image on a photodetecting surface 21a of the image sensor 21, a holder 23 which holds the image sensor 21 and the lens barrel 22, and a circuit board 24 which is mounted with the image sensor 21.

The holder 23 holds the lens barrel 22 so that it is movable along the optical axis B of the imaging optical system, and the position of the image sensor 21 relative to the imaging optical system can be adjusted by a movement of the lens barrel 22. The lens barrel 22 is fixed to the holder 23 with adhesive or the like after positioning of the image sensor 21.

The holder 23 is disposed in a housing hole which is formed in a tip hard portion 25 made of a metal material such as stainless steel, and is fixed to the tip hard portion 25. A tip portion of the treatment tool channel 14 and the illumination optical system are also disposed in respective housing holes formed in the tip hard portion 25, and are fixed to the tip hard portion 25.

In a state that the holder 23 is fixed to the tip hard portion 25, the optical axis B of the imaging optical system which is housed in the lens barrel 22 is approximately parallel with the longitudinal axis A of the insertion unit 6 and the photodetecting surface 21a of the image sensor 21 on which a subject image is to be formed by the imaging optical system is approximately perpendicular to the longitudinal axis A of the insertion unit 6.

Plural cables 27 are connected to the circuit board 24 which is mounted with the image sensor 21. The circuit board 24 and the image sensor 21 mounted on it are connected to the processor unit 4 by the cables 27.

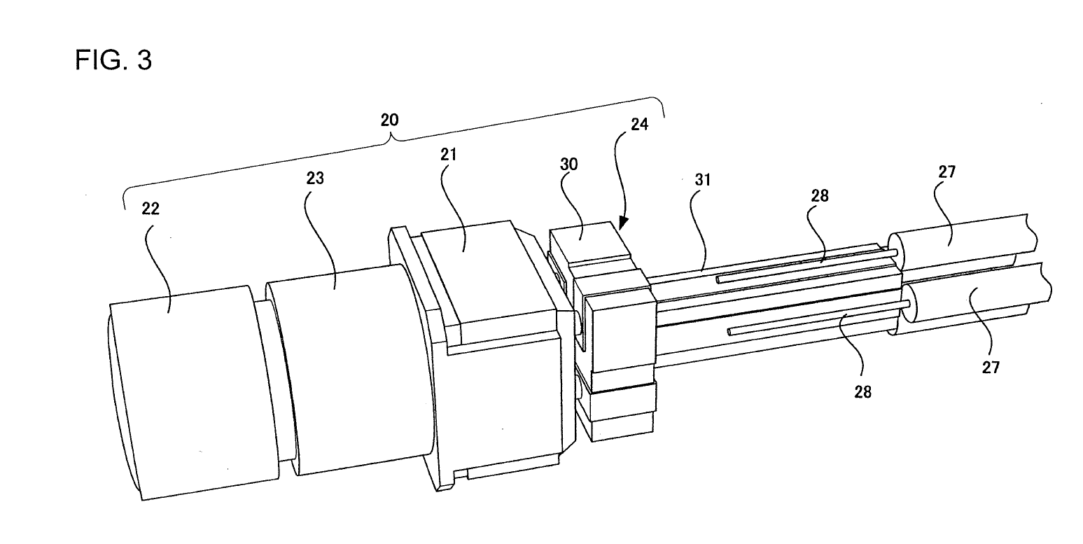

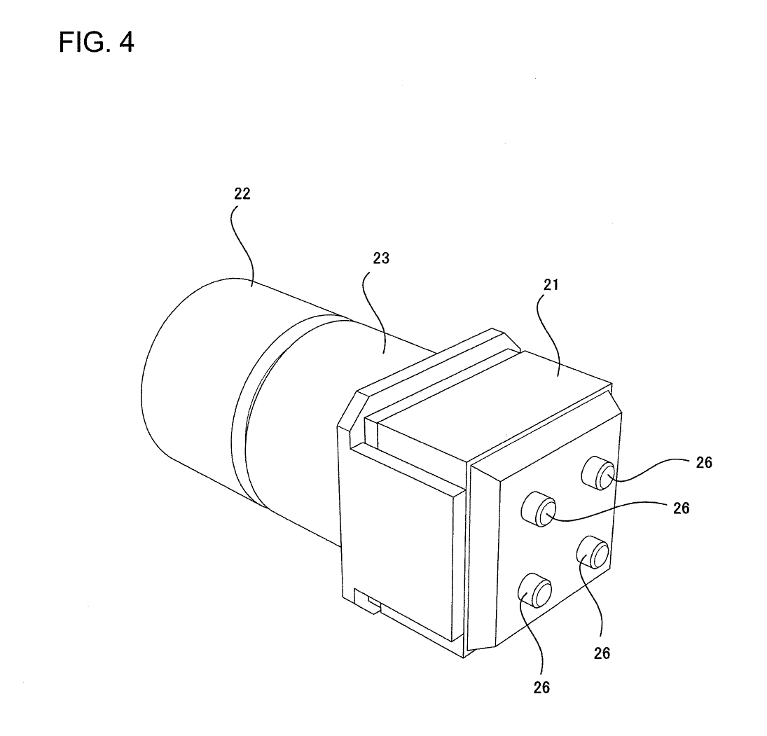

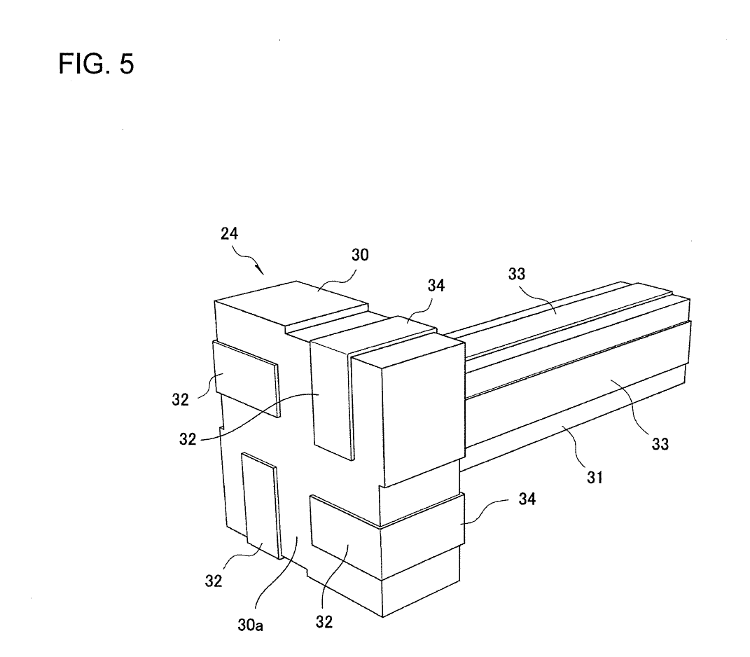

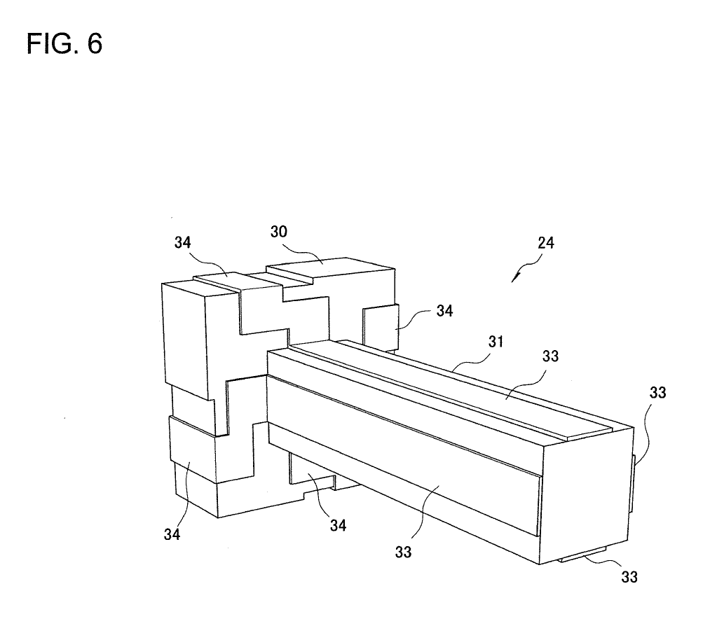

FIGS. 3-7 show the configuration of the imaging apparatus 20. Four terminals 26 are formed on the back surface, opposite to the photodetecting surface 21a, of the image sensor 21. Examples of these terminals are a video terminal for output of a video signal, a control terminal to receive a control signal for controlling the operation of the image sensor 21, a power terminal to receive operation power for the image sensor 21, and a ground terminal.

In the illustrated example, the terminals 26 are surface-mounting electrode pads such as solder balls. Alternatively, the terminals 26 may be pin terminals, for example. In general, surface-mounting electrode pads such as solder balls are more suitable for high-density mounting than pin terminals. The use of surface-mounting electrode pads is suitable in a case that the image sensor 21 is a very small one such as one whose outline is smaller than or equal to a 1-mm square, for example, and the area of the back surface on which the terminals 26 are formed is small.

The circuit board 24 has a sensor mounting portion 30 and a cable connection portion 31 which is integrated with the sensor mounting portion 30. The sensor mounting portion 30 is like a rectangular plate and faces the back surface of the image sensor 21 which is formed with the terminals 26. A mounting surface 30a, opposed to the image sensor 21, of the sensor mounting portion 30 is formed with four sensor connection lands 32 which are connected to the four respective terminals 26 of the image sensor 21.

The cable connection portion 31 is shaped like a rectangular prism and erected from the back surface, opposite to the mounting surface 30a, of the sensor mounting portion 30. The cable connection portion 31 which is erected from the sensor mounting portion 30 which is disposed approximately perpendicularly to the longitudinal axis A of the insertion unit 6 so as to face the back surface of the image sensor 21 extends parallel with the longitudinal axis A.

The side surfaces, extending in the longitudinal axis A of the insertion unit 6, of the cable connection portion 31 are formed with four cable connection lands 33 which are electrically continuous with the four sensor connection lands 32 of the sensor mounting portion 30 via circuit patterns 34, respectively. Conductors 28 of the cables 27 are soldered to the respective cable connection lands 33.

The circuit board 24 can be formed using a resin material such as glass epoxy. The sensor connection lands 32, the cable connection lands 33, and the circuit patterns 34 which electrically connect the lands 32 and 33 can be formed using an MID (molded interconnect device) technique for forming a metal foil pattern on the surface of a resin mold.

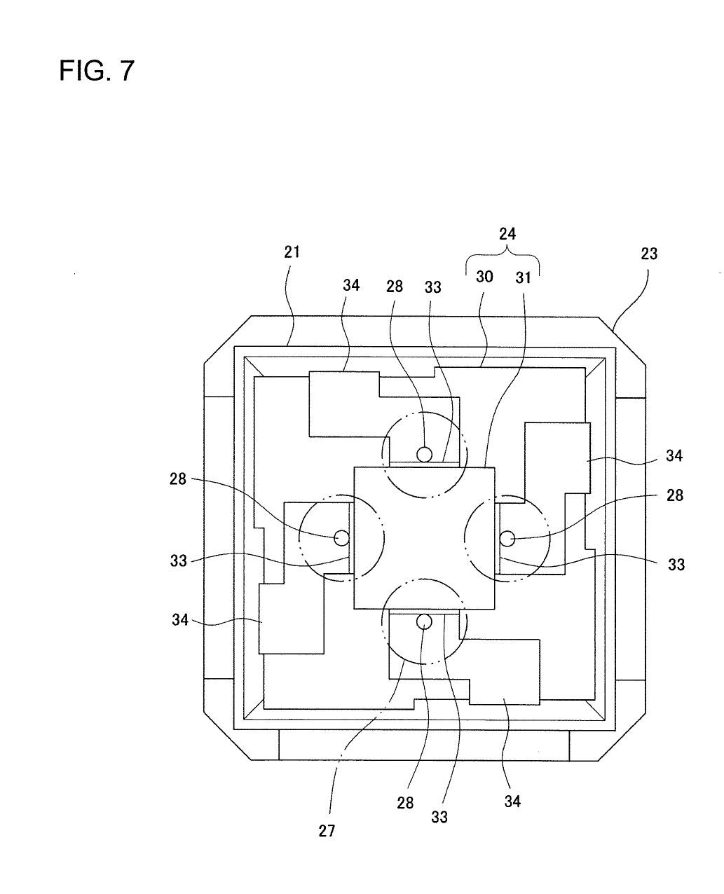

As shown in FIG. 7, when viewed from a distant point on the axis of the cable connection portion 31, or from a distant point on the longitudinal axis A of the insertion unit 6, the outline of the cable connection portion 31 is smaller than that of the sensor mounting portion 30 and the cable connection portion 31 is contained in the outer circumference of the sensor mounting portion 30.

The cable connection lands 33 of the cable connection portion 31 are formed on (distributed to) plural respective side surfaces of the rectangular-prism-shaped cable connection portion 31. As a result, the area of each cable connection land 33 can be made wider than in a case that all of the cable connection lands 33 are formed on one side surface of the cable connection portion 31, whereby the durability of the connections between the cable connection lands 33 and the cables 27 can be increased.

As long as the cable connection lands 33 of the cable connection portion 31 are formed on (distributed to) plural respective side surfaces of the cable connection portion 31, plural cable connection lands 33 may be formed on one side surface of the cable connection portion 31. However, in the illustrated example in which the cable connection portion 31 is shaped like a rectangular prism for formation of the four cable connection lands 33, it is preferable that only one cable connection land 33 be formed on each side surface. With this measure, the areas of the respective cable connection lands 33 can be increased effectively using all of the side surfaces of the cable connection portion 31. Only one cable connection land 33 can be formed on one or each side surface of the cable connection portion 31 if the cable connection portion 31 is shaped, in a sectional view, like a polygon the number of whose sides is larger than or equal to the number of cable connection lands 33.

Since the cable connection portion 31 is smaller than the sensor mounting portion 30 when viewed from a distant point on the axis of the cable connection portion 31, the cables 27 that are connected to the cable connection portion 31 can be contained in the outer circumference of the sensor mounting portion 30 or, even if portions of the cables 27 stick out of the outer circumference of the sensor mounting portion 30, those portions can be made small. As a result, the insertion unit 6 can be made thinner.

Since sensor mounting portion 30 is larger than the cable connection portion 31 when viewed from a distant point on the axis of the cable connection portion 31 and the sensor connection lands 32 are formed on the mounting surface 30a of the sensor mounting portion 30 whose outline coincides with that of the sensor mounting portion 30, the area of each sensor connection land 32 can be made wider than in a case that the sensor connection lands 32 are formed on one end surface, in the axial direction, of the cable connection portion 31 or an end portion of one side surface of the cable connection portion 31, whereby the durability of the connections between the sensor connection lands 32 and the terminals 26 of the image sensor 21 can be increased.

From the viewpoint of thinning of the insertion unit 6, it is preferable that when viewed from a distant point on the axis of the cable connection portion 31, or from a distant point on the longitudinal axis A of the insertion unit 6, the outline of the sensor mounting portion 30 be smaller than that of the image sensor 21 and the sensor mounting portion 30 be contained in the outer circumference of the image sensor 21.

The above-described configuration of the imaging apparatus 20 is particularly useful in a case that the image sensor 21 is a very small one such as one whose outline is smaller than or equal to a 1-mm square, for example, and the circuit board 24 is also a very small one accordingly.

Although the image sensor 21 has been described above as having the four terminals 26 on its back surface, the number of terminals 26 is not limited to four. For example, a total of five terminals 26, that is, the above-described four terminals 26 (e.g., video terminal, control terminal, power terminal, and ground terminal) plus a fifth terminal 26, may be formed. Examples of the fifth terminal 26 are an external clock terminal for receiving an external clock signal and a reset terminal for receiving a reset signal. A total of six terminals 26 may be formed by adding both of an external clock terminal and a reset terminal.

The sensor connection lands 32 of the sensor mounting portion 30 and the cable connection lands 33 of the cable connection portion 31 are provided in the same number as the number of terminals 26 of the image sensor 21. Where the number of cable connection lands 33 is five, only one cable connection land 33 can be formed on one or each side surface of the cable connection portion 31 if the cable connection portion 31 is shaped like a polygonal prism having five or more side surfaces. Where the number of cable connection lands 33 is six, only one cable connection land 33 can be formed on one or each side surface of the cable connection portion 31 if the cable connection portion 31 is shaped like a polygonal prism having six or more side surfaces.

This specification discloses an endoscope imaging apparatus which is installed in a tip portion of an insertion unit of an endoscope, comprising an image sensor having a photodetecting surface which crosses the longitudinal axis of the insertion unit; and a circuit board which is mounted with the image sensor and comprises a sensor mounting portion which has plural sensor connection lands to which respective terminals of the image sensor are connected and faces a back surface, opposite to the photodetecting surface, of the image sensor; and a polygonal-prism-shaped cable connection portion which has plural cable connection lands being electrically continuous with the respective sensor connection lands and extends parallel with the longitudinal axis, wherein an outline of the cable connection portion is smaller than an outline of the sensor mounting portion when viewed from a distant point on the axis of the cable connection portion, and the cable connection lands are formed on plural side surfaces of the cable connection portion in a distributed manner.

The disclosed endoscope imaging apparatus may be such that only one cable connection land is formed on one or each side surface of the cable connection portion.

The disclosed endoscope imaging apparatus may be such that when viewed from a distant point on the axis of the cable connection portion, an outline of the sensor mounting portion is smaller than an outline of the image sensor.

The disclosed endoscope imaging apparatus may be such that the terminals of the image sensor are electrode pads and are formed on the back surface of the image sensor.

The disclosed endoscope imaging apparatus may be such that when viewed from a distant point on a normal to the photodetecting surface of the image sensor, an outline of the image sensor is smaller than or equal to a 1-mm square.

The specification also discloses an endoscope comprising any of the above endoscope imaging apparatus.

Although the invention has been described above in relation to preferred embodiments and modifications thereof, it will be understood by those skilled in the art that other variations and modifications can be effected in these preferred embodiments without departing from the scope and spirit of the invention.

What is claimed is:

1. An endoscope imaging apparatus which is installed in a tip portion of an insertion unit of an endoscope, comprising:
an image sensor having a photodetecting surface which crosses a longitudinal axis of the insertion unit; and
a circuit board which is mounted with the image sensor and comprises:
a sensor mounting portion which has plural sensor connection lands to which respective terminals of the image sensor are connected and faces a back surface, opposite to the photodetecting surface, of the image sensor; and
a polygonal-prism-shaped cable connection portion which has plural cable connection lands being electrically continuous with the respective sensor connection lands and extends parallel with the longitudinal axis,
wherein an outline of the cable connection portion is smaller than an outline of the sensor mounting portion when viewed from a distant point on an axis of the cable connection portion, and the cable connection lands are formed on plural side surfaces of the cable connection portion in a distributed manner.

2. The endoscope imaging apparatus according to claim 1, wherein only one cable connection land is formed on one or each side surface of the cable connection portion.

3. The endoscope imaging apparatus according to claim 1, wherein when viewed from a distant point on the axis of the cable connection portion, an outline of the sensor mounting portion is smaller than an outline of the image sensor.

4. The endoscope imaging apparatus according to claim 2, wherein when viewed from a distant point on the axis of the cable connection portion, an outline of the sensor mounting portion is smaller than an outline of the image sensor.

5. The endoscope imaging apparatus according to claim 3, the terminals of the image sensor are electrode pads and are formed on the back surface of the image sensor.

6. The endoscope imaging apparatus according to claim 4, the terminals of the image sensor are electrode pads and are formed on the back surface of the image sensor.

7. The endoscope imaging apparatus according to claim 1, wherein when viewed from a distant point on a normal to the photodetecting surface of the image sensor, an outline of the image sensor is smaller than or equal to a 1-mm square.

8. The endoscope imaging apparatus according to claim 2, wherein when viewed from a distant point on a normal to the photodetecting surface of the image sensor, an outline of the image sensor is smaller than or equal to a 1-mm square.

9. The endoscope imaging apparatus according to claim 3, wherein when viewed from a distant point on a normal to the photodetecting surface of the image sensor, an outline of the image sensor is smaller than or equal to a 1-mm square.

10. The endoscope imaging apparatus according to claim 4, wherein when viewed from a distant point on a normal to the photodetecting surface of the image sensor, an outline of the image sensor is smaller than or equal to a 1-mm square.

11. The endoscope imaging apparatus according to claim 5, wherein when viewed from a distant point on a normal to the photodetecting surface of the image sensor, an outline of the image sensor is smaller than or equal to a 1-mm square.

12. The endoscope imaging apparatus according to claim 6, wherein when viewed from a distant point on a normal to the photodetecting surface of the image sensor, an outline of the image sensor is smaller than or equal to a 1-mm square.

13. An endoscope comprising the endoscope imaging apparatus according to claim 1.
